(19) United States
(12) Patent Application Publication
(10) Pub. No.: US 2021/0085764 A1
(43) Pub. Date: Mar. 25, 2021

(54) DRY POWDER FORMULATIONS OF ALPHA-1 ANTITRYPSIN

(71) Applicant: KAMADA LTD., Nes Ziona (IL)

(72) Inventor: Liliana BAR, Rehovot (IL)

(21) Appl. No.: 16/472,732

(22) PCT Filed: Dec. 20, 2017
(86) PCT No.: PCT/IL2017/051367

§ 371 Date: Jun. 21, 2019

Related U.S. Application Data
(60) Provisional application No. 62/437,675, filed on Dec. 22, 2016.

(51) Int. Cl. A61K 38/57; A61K 9/16; A61K 9/00; A61K 45/06

(52) U.S. Cl. CPC A61K 38/57; A61K 9/1623; A61K 45/06; A61K 9/0075; A61K 9/1617

(57) ABSTRACT

Dry powder formulations comprising AAT and methods of using thereof are provided. Said formulations comprise AAT molecules in their monomelic form and excipients which are highly suitable for inhalation administering.

### FIELD OF THE INVENTION

The present invention relates to dry powder formulations of alpha-1 antitrypsin. In particular, the present invention relates to dry powder formulations having a high concentration of monomeric alpha-1 antitrypsin; and specific excipients, and methods of using same.

### BACKGROUND OF THE INVENTION

Numerous methods have been devised for delivering active ingredients into living organisms. Traditional oral therapeutic dosage forms include both solids (tablets, capsules, pills, etc.) and liquids (solutions, suspensions, emulsions, etc.). Parenteral dosage forms include solids and liquids as well as aerosols (administered by inhalers, etc.), injectables (administered with syringes, micro-needle arrays, etc.), topicals (foams, ointments, etc.), and suppositories, among other dosage forms. These dosage forms are more effective in delivering low molecular weight drugs, but are less effective with high molecular weight ingredients when controlling either the spatial or the temporal component of the active ingredient distribution is more difficult. Oral drug delivery is perhaps the most convenient method, but many drugs are degraded in the digestive tract before they can be absorbed. This is particularly true for bio-therapeutics, i.e. pharmaceutically active peptides (e.g. growth factors), proteins (e.g. enzymes, antibodies), oligonucleotides (e.g. RNA, DNA, PNA), hormones and other natural substances or similar synthetic substances. Intravenous infusion and subcutaneous injection is frequently an effective route for systemic drug delivery, including the delivery of proteins, but enjoys a low patient acceptance. Since the need to inject drugs on a frequent schedule, such as insulin one or more times a day, can be a source of poor patient compliance, a variety of alternative routes of administration have been developed, including transdermal, intranasal, intrarectal, intravaginal, and pulmonary delivery.

Oral inhalation (pulmonary delivery) is a common method of drug delivery. The pulmonary drug dispersion compositions are designed to be delivered to the patient by inhalation so that the active ingredient can reach the different regions of the lungs. Pulmonary delivery is particularly useful for the delivery of macromolecules such as proteins which are difficult to be delivered to the lungs by other routes of administration. Such pulmonary delivery can be effective both for systemic delivery and for localized delivery to treat diseases of the lung.

The ability to deliver pharmaceutical macromolecule compositions as dry powders is challenging in certain aspects. For example, the dosage of pharmaceutical compositions is often critical, so it is desirable that dry powder delivery systems be able to deliver an accurate and reliable intended amount of drug. In addition, many pharmaceutical compositions are quite expensive, necessitating maximum utilization. Thus, the ability to efficiently formulate, process, package, and deliver the dry powders with a minimal loss of drug is critical. Dry powder formulation of high molecular weight molecules, particularly proteins, pose the challenge of obtaining high amounts of the protein (in the range of tens milligram and not the typical range of micrograms) with very low excipient amount to maximize the protein content in each dosage form while keeping low water content and minimal percentage of protein aggregates.

While the permeability of natural macromolecules in the lung is well known, the combined inefficiencies of macromolecule production processes and macromolecule delivery has limited commercialization of dry macromolecule powders for pulmonary delivery.

The use of spray drying for the preparation of dry particles from liquid starting materials is a conventional process, but has usually been limited to small molecules and other stable materials that are less sensitive to thermal and other rigorous treatments. Macromolecule compositions, such as proteins are often labile and subject to degradation and/or aggregation during the spray drying process. For efficient and safe pulmonary delivery, it is desirable that the particles size be maintained below 5 μm and in a highly homogeneous form. Larger particle sizes easily stick in the mouth and throat. In addition, it is necessary to obtain dry powder as pure as possible and in a useable form.

Powders with a particle size suitable for inhalation have a tendency of aggregating. These aggregated forms need to be de-aggregated before entering the airways of the user. De-aggregation can be achieved by introducing energy e.g.

electrical, mechanical, or aerodynamic energy. Various formulations and techniques are used to produce a uniformed and non aggregated powder. For example, there are techniques that include modification of the particles shape and surface properties e.g. controlled forming of powder pellets, as well as addition of inert carrier. The formulations usually contain high concentrations of excipients intended to stabilize and reduce the aggregation and degradation of the active agent.

### Alpha-1 Antitrypsin

Alpha-1 Antitrypsin (AAT), also known as Alpha-1-Proteinase Inhibitor (API), is a plasma protein belonging to the family of serine proteinase inhibitors. The natural protein is a glycoprotein having an average molecular weight of 50,600 daltons, produced primarily by the liver and secreted into the circulatory system. The protein is a single polypeptide chain, to which several oligosaccharide units are covalently bound in three N-glycosylation sites. AAT has a role in controlling tissue destruction by endogenous serine proteinases, and is the most prevalent serine proteinase inhibitor in blood plasma. AAT inhibits, inter alia, trypsin, chymotrypsin, various types of elastases, skin collagenase, renin, urokinase and proteases of polymorphonuclear lymphocytes.

Plasma derived AAT (pAAT) is currently used therapeutically for the treatment of pulmonary emphysema in patients who have a genetic AAT deficiency, also known as Alpha-1 Antitrypsin Deficiency or Congenital Emphysema. Purified pAAT has been approved for replacement therapy (also known as “augmentation therapy”) in these patients. There is a continuous effort targeted at producing recombinant AAT, but as of today there is no approved recombinant product. The endogenous role of AAT in the lungs is predominantly to regulate the activity of neutrophil elastase, which breaks down foreign proteins present in the lung. In the absence of sufficient quantities of AAT, the elastase breaks down lung tissue, which over time results in chronic lung tissue damage and emphysema.

AAT has also been proposed as a treatment for cystic fibrosis (CF) patients who suffer from recurrent endobronchial infections and sinusitis. The major cause of morbidity and mortality among CF patients is lung diseases. CF patients carry a mutation in the CFTR gene, resulting in a malfunctioning CTFR protein, defective water and salt transport and the ensuing thick secretions in the lung.

The membrane defect caused by the CFTR mutation leads to chronic lung inflammation and infection. In normal individuals, elastase secreted by neutrophils in response to infection is neutralized by AAT. AAT is known to penetrate into pulmonary tissue and exert its activity within this tissue. In patients with CF, however, the unregulated inflammatory response overwhelms the normal protease (elastase)/antiprotease (AAT) balance. The abnormal cycle is destructively self-perpetuating and leads to the accumulation of elastase in the lung and ultimately to tissue damage, destruction of the lung architecture, severe pulmonary dysfunction and, ultimately, death. Supplemental AAT may reduce the deleterious effects associated with excessive amounts of elastase.

International application WO 2005/027821 to the Applicant of the present invention teaches a novel composition of purified, stable, active alpha-1 antitrypsin for intravenous administration and inhalation, and process for its preparation. That application teaches an aerosol formulation comprising about 10% to about 20% AAT.

AAT is currently administered intravenously. For example, the Glassia®, Aralast®, Zemaira® and Prolastin® brands of human Alpha-1-Proteinase Inhibitor are intravenous formulations indicated for augmentation therapy in patients having congenital deficiency of AAT with clinically evident emphysema. An AAT formulation for efficient administration in inhalation is highly desired, and not yet commercially available due to problems in achieving suitable quantity, dispersion and activity of the protein. International Application WO 2005/048985 discloses compositions comprising AAT, which further comprise a stabilizing carbohydrate, a surfactant and an antioxidant to stabilize the AAT for use as a therapeutic, wherein the composition is preferably formulated to be administered by inhalation.

International application WO 01/34232, discloses an inhalation nebulizer providing an increased amount of aerosol during inhalation while minimizing both aerosol losses during exhalation and the residual drug in the nebulizer reservoir. The nebulizer includes an aerosol generator that atomizes the liquid through a vibrating diaphragm into particle sizes that are efficiently delivered to the lungs. This nebulizer is currently commercialized under the trade name eFlow®. Classic jet and ultrasonic nebulizers have the disadvantage of potentially denaturizing the active agent by high shear forces (jet and ultrasonic nebulizers) and temperature increase (ultrasonic nebulizers). eFlow® incorporates a “gentle” aerosolization mechanism that minimizes exposure of the drug to shear stresses by reducing the shear stresses and the residence time in the shear fields and does not heat the liquid formulation. International Applications WO 03/026832; WO 2004/014569; WO 2004/052436 and U.S. Pat. No. 5,518,179 disclose further aspects of the eFlow® technology. European Patent No. 1981572 discloses a highly efficient system for treating pulmonary diseases combining the nebulizer disclosed in WO 01/34232 and the ready-to-use AAT solution disclosed in WO 2005/027821.

U.S. Pat. No. 6,655,379 discloses a method and device for the pulmonary delivery of an active agent formulation where inspiratory flow rate of the active agent formulation is less than 17 liters/min. The active agent formulation may be provided in dry powder, in nebulized form, or in the form of aerosolized particles in admixture with a propellant. That invention is exemplified in conjugation with inhalable insulin powder.

U.S. Pat. No. 6,881,398 discloses a therapeutic dry powder preparation and a method of administering such a preparation.

U.S. Pat. No. 8,173,168 discloses a process for preparing ultrafine powders of biological macromolecules comprises atomizing liquid solutions of the macromolecules, drying the droplets formed in the atomization step, and collecting the particles which result from drying.

There remains an unmet need for dry powder compositions having high percent of AAT in its active monomeric form while exhibiting stability and low aggregation level.

### SUMMARY OF THE INVENTION

The present invention according to some aspects provides highly dispersible dry powder compositions comprising high concentration of active alpha-1 antitrypsin (AAT) and specific excipients, suitable for pulmonary delivery of AAT. The dry powder compositions disclosed herein comprise according to some embodiments AAT molecules in their monomeric form, having low aggregation level. The AAT dry powder compositions disclosed herein exhibit an exceptional stability and low aggregation properties, and thus are highly suitable for use with inhalation devices as well as in other dry-powder dosage forms.

The present invention is based in part on the unexpected discovery that a spray drying process of high concentrations of AAT admixed with various specific excipients results in homogenous dry powder which is highly suitable for delivery by inhalation.

Without being bound by any particular theory or mechanism of action, the combination of the AAT with particular excipients including Trehalose, Glycine, Dipalmitoylphosphatidylcholine (DPPC), and Ectoin, maintains the AAT molecules in their monomeric state and enable the preparation of uniformly, highly concentrated AAT dry powder.

According to one aspect, the present invention provides a dry powder composition comprising at least 60% alpha-1 antitrypsin (AAT) and at least one excipient selected from the group consisting of Trehalose, Glycine, Dipalmitoylphosphatidylcholine (DPPC), and Ectoin, wherein at least 90% of the AAT is in a monomeric form.

According to certain embodiments, the dry powder composition comprising at least 60%, 65%, 70%, 71%, 72%, 73%, 74%, 75%, 76%, 77%, 78%, 79%, 80%, 8%1, 82%, 83%, 94%, 85%, 86%, 87%, 88%, 89%, 90%, 91%, 92%, 93%, 94%, 95%, 96%, 97%, 98% or 99% alpha-1 antitrypsin (AAT).

According to certain exemplary embodiments, the dry powder composition comprising at least 60% AAT.

According to certain exemplary embodiments, the dry powder composition comprising at least 75% AAT.

According to additional exemplary embodiments, the dry powder composition comprising at least 80% AAT.

According to further additional exemplary embodiments, the dry powder composition comprising at least 85% AAT.

According to some embodiments, the excipient is DPPC. According to certain embodiments, the excipient is Ectoin. According to additional embodiments, the excipient is a combination of Glycine and Trehalose.

According to some embodiments, the dry powder composition is suitable for delivery to the lung by inhalation.

According to some embodiments, the non-monomeric forms percent (NMF %) of the dry powder is less than 8%. According to other embodiments, the non-monomeric forms percent (NMF %) of the dry powder is less than 7.5, 7, 6.5, 6, 5.5, or 5%. Each possibility represents a separate embodiment of the invention. According to certain embodiments, the non-monomeric particles consist essentially of AAT.

According to some embodiments, the particle size distribution of the dry powder, measured as D₅₀(μm), is less than 10 μm. According to some embodiments, the particle size distribution of the dry powder, measured as D₅₀(μm) after 1 month of storage, is less than 10 μm. According to other embodiments, the particle size distribution of the dry powder, measured as D₅₀(μm) after 1 month of storage, is less than 5.5μm.

According to some embodiments, the particle size distribution of the dry powder, measured as D₉₀(μm) after 1 month of storage, is less than 10 μm. According to certain embodiments, the particle size distribution of the dry powder, measured as D₉₀(μm) after 1 month of storage, is less than 15 μm.

According to some embodiments, the dry powder is prepared by a spray-drying method.

According to some embodiments, the dry powder composition has moisture content below 10% by weight. According to certain embodiments, the dry powder composition has moisture content below 10, 9, 8, 7, 6, or 5% by weight.

According to some embodiments, an inhalation device is provided comprising the dry powder composition.

According to some embodiments, the dry powder has an emitted dose of at least 60%. According to certain embodiments, the dry powder has an emitted dose of at least about 60%, 65%, 70%, 75%, 80%, 85%, or 90%. Each possibility represents a separate embodiment of the invention.

According to some embodiments, the dry powder is for use in treating a subject in a need for AAT supplement.

According to some embodiments, the dry powder is for use in treating a disease or condition selected from the group consisting of: AAT-deficiency, a disease associated with AAT-deficiency, and a disease that would benefit from AAT administration. According to certain embodiments, the disease is selected from the group consisting of emphysema; chronic obstructive pulmonary disease (COPD); bronchiectasis; parenchymatic and fibrotic lung diseases or disorders; cystic fibrosis, interstitial pulmonary fibrosis and sarcoidosis; tuberculosis; and lung diseases and disorders secondary to HIV. Each possibility represents a separate embodiment of the invention.

According to an additional aspect, the present invention provides a method of treating a subject in need thereof, the method comprising administering to the subject in need thereof via inhalation a therapeutically effective amount of the dry powder of the invention.

According to certain embodiments, the dry powder is administered to the lungs of the subject.

According to some embodiments, the subject has AAT deficiency. According to certain exemplary embodiments, the subject suffers from emphysema secondary to AAT deficiency.

According to some embodiments, the subject suffers from a disease selected from the group consisting of emphysema; chronic obstructive pulmonary disease (COPD); bronchiectasis; parenchymatic and fibrotic lung diseases or disorders; cystic fibrosis, interstitial pulmonary fibrosis and sarcoidosis; tuberculosis; and lung diseases and disorders secondary to HIV. Each possibility represents a separate embodiment of the invention. According to certain embodiments, the disease is chronic obstructive pulmonary disease (COPD). According to other embodiments, the disease is cystic fibrosis.

According to some embodiments, the subject is a human subject.

According to some embodiments, the dry powder is administered in a regime of at least once a week.

According to some embodiments, the treating is in combination with at least one additional therapy. According to certain embodiments, the additional therapy is selected from the group consisting of antibiotic therapy, administration of bronchodilators and anti-inflammatory therapy other than AAT therapy.

Other objects, features and advantages of the present invention will become clear from the following description, examples and drawings.

### BRIEF DESCRIPTION OF THE FIGURES

FIG. 1 demonstrates the toxicokinetic (TK) results of the two AAT powder formulations.

### DETAILED DESCRIPTION OF THE INVENTION

The present invention is directed, inter alia, to dry powder compositions of AAT. The dry powder compositions preserve the AAT molecules in their monomeric form and exhibit high stability. Unexpectedly, a combination of high concentrations of AAT with specific excipients such as Trehalose, Glycine and DPPC, exhibits superior properties, making it suitable for pulmonary administration. It is to be particularly understood that while the dry powder compositions of the present invention are intended primarily for pulmonary administration, other AAT dry-powder formulation are also encompassed within the scope of the present invention.

As used herein, the term “Alpha-1 Antitrypsin” (AAT) refers to a glycoprotein which is naturally produced by the liver and secreted into the circulatory system. AAT belongs to the Serine Proteinase Inhibitor (Serpin) family of proteolytic inhibitors. This glycoprotein consists of a single polypeptide chain containing one cysteine residue and 12-13% of the total molecular weight of carbohydrates. AAT has three N-glycosylation sites at asparagine residues 46, 83 and 247, which are occupied by mixtures of complex bi- and triantennary glycans. This gives rise to multiple AAT isoforms, having isoelectric point in the range of 4.0 to 5.0. The glycan monosaccharides include N-acetylglucosamine, mannose, galactose, fucose and sialic acid. AAT serves as a pseudo-substrate for elastase; elastase attacks the reactive center loop of the AAT molecule by cleaving the bond between methionine358-serine359 residues to form an AAT-elastase complex. This complex is rapidly removed from the blood circulation. AAT is also referred to as “alpha-1 Proteinase Inhibitor” (API). The term “glycoprotein” as used herein refers to a protein or peptide covalently linked to a carbohydrate. The carbohydrate may be monomeric or composed of oligosaccharides.

The AAT can be of a variety of different forms, including purified naturally occurring AAT and a recombinant AAT.

The term “dry powder” refers to a powder composition that contains finely dispersed dry particles that are capable of being dispersed in an inhalation device and subsequently inhaled by a subject.

The particles of the dry powder composition have particle size distribution that enables the particles to target the alveolar region of the lung when delivered via inhalation. The particle-size distribution (PSD) of a powder is a list of values or a mathematical function that defines the relative amount of particles present according to size. The powders of the invention are generally polydispersed (i.e., consist of a range of particle sizes). In particular embodiments, the term “particle size distribution” refers to the size distribution of particle system and represents the number of solid particles that fall into each of the various size ranges, given as a percentage of the total solids of all sizes in the sample of interest.

As used herein, the term “particle size distribution D₉₀ value” is defined as the numerical value, expressed in microns, at which 90 percent of the particles have particle sizes which are less than or equal to that value. As used herein, the term “particle size distribution D₅₀ value” is defined as the numerical value, expressed in microns, at which 50 percent of the particles have particle sizes which are less than or equal to that value.

According to some embodiments, the average particle size is below 10 μm. In other embodiments, the average particle size is below 9, 8, or 7 μm. Each possibility represents a separate embodiment of the invention. In additional embodiments, the average particle size is between 1 to10 μm, 2 to 9 μm, 3 to 8 μm, 4 to 8 μm, or 4-8 μm. Each possibility represents a separate embodiment of the invention. The average particle size of the powder may be measured as mass mean diameter (MMD) by conventional techniques.

The term “dry” means that the particles of the powder have moisture content such that the powder is physically and chemically stable in storage at room temperature. According to some embodiments, the moisture content of the particles is below 10%, 8%, 6%, 4%, 2% or 1% by weight. Each possibility represents a separate embodiment of the invention.

A dry powder that is “suitable for pulmonary delivery” refers to a composition comprising solid or partially solid particles that are capable of being (i) readily dispersed by an inhalation device and (ii) inhaled by a subject so that a portion of the particles reach the lungs to permit penetration into the alveoli. Such a powder is considered to be “respirable”.

The dry powder composition may be incorporated into unit dosage form within an inhalation device. The amount depends on various factors and can be determined according to, without limiting, the disease to be treated, the target population, and inhalation device.

The term “Emitted dose” is an indication of the delivery of a drug formulation from a specific inhaler device after a dispersion event. More specifically, emitted dose is a measure of the percentage of powder which exits from a unit dose package.

A “dispersible” or “dispersive” powder is one having an emitted dose value of at least 60%, 70%, 75%, 80%, 85%, or 90%. Each possibility represents a separate embodiment of the invention.

Because the powders are dispersible, it is highly preferred that they be manufactured in a unit dosage form in a manner that allows for ready manipulation by the formulator and by the consumer. According to some embodiments, the unit dosage weight between 0.2-40 mg, 10-40 mg, or 20-40 mg. Each possibility represents a separate embodiment of the invention.

The present invention now discloses a dry powder composition characterized in that the majority of the composition mass is composed of AAT. Furthermore, most of the AAT is in its monomeric form. The-AAT-comprising dry powder of the invention is advantageous over hitherto known dry powder compositions comprising proteinaceus macromolecules, particularly AAT in that the AAT is in its active monomer form and in that the total amount of the composition to be inhaled is close to the therapeutic amount of AAT.

According to some embodiments, the amount of AAT is above 60% by weight of the dry powder. According to other embodiments, the amount of AAT is above 70% by weight of the dry powder. According to further embodiments, the amount of AAT is above 80% by weight of the dry powder. According to certain embodiments, the amount of AAT is between 60% and 95% by weight of the dry powder. According to additional embodiments, the amount of AAT is between 80% and 95% by weight of the dry powder. According to further embodiments, the amount of AAT is between 85% and 95% by weight of the dry powder.

The powders of the invention may further be characterized by their densities. According to some embodiments, the dry powder comprises particles having a bulk density from 0.1 to 10 grams per cubic centimeter. According to certain embodiments, the dry powder comprises particles having a bulk density from 0.1 to 2 grams per cubic centimeter. According to additional embodiments, the dry powder comprises particles having a bulk density from 0.15 to 1.5 grams per cubic centimeter.

An additional measure for characterizing the dry powder is the none monomeric forms percent (NMF %), which implies for the aggregation level of the powder after reconstitution as measured by SEC-HPLC. According to some embodiments, the NMF % of the reconstituted powder is less than 8%. According to certain embodiments, the NMF % of the reconstituted powder is less than 7%. According to additional embodiments, the NMF % of the reconstituted powder is less than 6%.

The compositions described herein also possess good stability with respect to both chemical stability and physical stability, i.e., aerosol performance, over time. Generally, with respect to chemical stability, the AAT contained in the formulation will degrade by no more than about 10% over a time course of three months, preferably by no more than about 7%, and more preferably by no more than 5%, upon storage of the composition under ambient conditions. As shown for the exemplary AAT formulations with respect to physical stability, after one month of storage at 40° C./75% RH no substantial deterioration of particle size distribution value, percentage of moisture, or appearance were found.

According to some embodiments, the dry powder composition has a degradation of less than about 20%, 15%. 10% or 5% by weight of the AAT upon storage of said composition under ambient conditions for a period of at least one month.

The term “degradation” as used herein refers to AAT protein which has been degraded. The term also refers to loss of function as a result of a structural conformation change.

The composition of the invention comprises specific pharmaceutical acceptable excipients. Without wishing to be bound by any specific theory or mechanism of action, the excipients of the present invention provide for, at least partially, the prevention of aggregation process and therefore enhanced dispersibility of the powder.

According to some embodiments, the formulation comprises an excipient selected from the group consisting of lactose, trehalose, glycine, ectoin, DPPC, and combinations thereof. According to certain embodiments, the excipient is trehalose. According to certain embodiments, the excipient is glycine. According to certain embodiments, the excipient is ectoin. According to certain embodiments, the excipient is DPPC. According to certain embodiments, the excipient is a combination of glycine and Mannitol. According to certain embodiments, the excipient is a combination of glycine and DPPC.

According to some embodiments, the excipient amount is up to 30% of the dry powder weight. According to some embodiments, the excipient amount is up to 25% of the dry powder weight. According to further embodiments, the excipient amount is up to 15% of the dry powder weight. According to yet further embodiments, the excipient amount is up to 20% of the dry powder weight. According to certain embodiments the excipient amount is up to 3%, 4%, 5%, 6%, 7%, 8%, 9%, 10%, 11%, 12%, 13%, 14%, 15%, 16%, 17%, 18%, 19%, 20%, 21%, 22%, 22%, 23%, 24%, 25%, 26%, 27%, 28%, 29%, or 30% of the dry powder weight. Each possibility represents a separate embodiment of the invention. According to additional embodiments, the excipient amount is between 5% to 30% of the dry powder weight. According to additional embodiments, the excipient amount is between 5: to 20% of the dry powder weight. According to additional embodiments, the excipient amount is between 5% to 15% of the dry powder weight. According to other embodiments, the excipient amount is between 10% to 15% of the dry powder weight. According to yet other embodiments, the excipient amount is between 8% to 12% of the dry powder weight.

“Pharmaceutically acceptable excipient or carrier” refers to an excipient that may optionally be included in the compositions of the invention, and taken into the lungs with no significant adverse toxicological effects to the subject, and particularly to the lungs of the subject.

According to certain embodiments, the dry powder composition of the present invention consists of AAT and at least one excipient selected from the group consisting of Trehalose, Glycine, Dipalmitoylphosphatidylcholine (DPPC), and Ectoin, wherein the AAT is at least 60% (w/w) of the composition and wherein at least 90% of the AAT is in a monomeric form.

According to some embodiments, the dry powder further comprises a pharmaceutical acceptable salt. According to some embodiment, the dry powder comprises up to 5% pharmaceutical acceptable salt. “Pharmaceutically acceptable salt” includes, but is not limited to, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including alkyl substituted ammonium).

The compositions of the invention may also include polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-.beta.-cyclodextrin and sulfobutylether-.beta.-cyclodextrin), polyethylene glycols, and pectin.

The compositions may further include flavoring agents, taste-masking agents, inorganic salts (e.g., sodium chloride), sweeteners, antioxidants, antistatic agents, surfactants (e.g., polysorbates such as “TWEEN 20” and “TWEEN 80”), sorbitan esters, lipids (e.g., phospholipids such as lecithin and other phosphatidylcholines, phosphatidylethanolamines), fatty acids and fatty esters, steroids (e.g., cholesterol), and chelating agents (e.g., EDTA, zinc and other such suitable cations). Other pharmaceutical excipients and/or additives suitable for use in the dry powder compositions are listed, for example, in “Remington: The Science & Practice of Pharmacy”, 22^{th} ed., Allen et al., (1995), and in the “Physician's Desk Reference”, 52.sup.nd ed., Medical Economics, Montvale, N.J. (1998).

### Dry Powder Preparation

The AAT starting material can be prepared as known in the art. Exemplary method of purifying AAT is described in U.S. Patent No. 7,879,800.

A fine particle size distribution powder may be prepared by any known method in the art (spray drying, micronization, and the like). According to some embodiments, a liquid formulation is spray-dried to produce a dry powder. Spray drying of the formulations is carried out, for example, as described generally in the “Spray Drying Handbook”, 5.sup.th ed., K. Masters, John Wiley & Sons, Inc., NY, N.Y. (1991), and in International application WO 97/41833.

Utilizing the spray-dried approach, the AAT is first dissolved in water, optionally containing a physiologically acceptable buffer. According to certain exemplary embodiments, the AAT is in a form of a ready-to-use sterile solution produced essentially according to the teachings of U.S. Pat. No. 7,879,800. The pH range of active agent-containing solutions is generally between about 4 and 11, with nearer neutral pHs being preferred, since such pHs may aid in maintaining the physiological compatibility of the powder after dissolution of powder within the lung. The aqueous formulation may optionally contain additional water-miscible solvents, such as acetone, alcohols and the like. Representative alcohols are lower alcohols such as methanol, ethanol, propanol, isopropanol, and the like. The pre-spray dried solutions will generally contain solids dissolved at a concentration from 0.01% (weight/volume) to about 40% (weight/volume), usually from 0.1% to 20% (weight/volume).

The solutions are then spray dried in a conventional spray drier, such as those available from commercial suppliers such as Niro A/S (Denmark), Buchi (Switzerland), Eurotherm (US) and the like, resulting in a dispersible, dry powder. Optimal conditions for spray drying the solutions will vary depending upon the formulation components, and are generally determined experimentally. The gas used to spray dry the material is typically air, although inert gases such as nitrogen or argon are also suitable. Moreover, the temperature of both the inlet and outlet of the gas used to dry the sprayed material is such that it does not cause decomposition of the active agent in the sprayed material. Such temperatures are typically determined experimentally, although generally, the inlet temperature will range from about 50° C. to about 200° C. while the outlet temperature will range from about 30° C. to about 150° C.

Alternatively, the composition may be prepared by spray-drying a suspension, as described in U.S. Pat. No. 5,976,574. In this method, the drug is dissolved in an organic solvent, e.g., methanol, ethanol, isopropanol, acetone, heptane, hexane chloroform, ether, followed by suspension of the hydrophilic excipient in the organic solvent to form a suspension. The suspension is then spray-dried to form particles. Exemplary solvents, for both of the above spray-drying methods include alcohols, ethers, ketones, hydrocarbons, polar aprotic solvents, and mixtures thereof.

The dry powders of the invention may also be prepared by combining aqueous solutions or suspensions of the formulation components and spray-drying them simultaneously in a spray-dryer, as described in U.S. Pat. No. 6,001,336.

Alternatively, powders may be prepared by lyophilization, vacuum drying, spray freeze drying, super critical fluid processing, air drying, or other forms of evaporative drying. According to certain preferred embodiments the dry powder formulation is provided in a form that possesses improved handling/processing characteristics, e.g., reduced static, better flowability, low caking, and the like, by preparing compositions composed of fine particle aggregates, that is, aggregates or agglomerates of the above-described dry powder particles, where the aggregates are readily broken back down to the fine powder components for pulmonary delivery, as described, e.g., in U.S. Pat. No. 5,654,007.

Dry powders may also be prepared by agglomerating the powder components, sieving the materials to obtain agglomerates, spheronizing to provide a more spherical agglomerate, and sizing to obtain a uniformly-sized product, as described, e.g., in WO 95/09616.

Dry powders may also be manufactured by additional processes well known in the art, e.g. by means of conventional mixing, dissolving, granulating, grinding, pulverizing, dragee-making, levigating or lyophilizing processes.

Once formed, the dry powder compositions are preferably maintained under dry (i.e., relatively low humidity) conditions during manufacture, processing, and storage. Irrespective of the drying process employed, the process will preferably result in respirable, highly dispersible particles comprising active AAT molecules.

Uses of the Dry Powder

The dry powder composition may be delivered using any suitable dry powder inhaler (DPI). When administered using a DPI device, the powder is contained in a receptacle having a puncturable lid or other access surface, where the receptable may contain a single dosage unit or multiple dosage units. Convenient methods for filling large numbers of cavities (i.e., unit dose packages) with metered doses of dry powder medicament are described, e.g., in WO 97/41031.

Other dry powder dispersion devices for pulmonary administration of dry powders include those described, for example, in EP Pat No. 129985, in EP Pat. No.472598, in EP Pat. No. 467172, and in U.S. Pat. No. 5,522,385. Also suitable for delivering the dry powders of the invention are inhalation devices such as the Astra-Draco “TURBUHALER”. This type of device is described in detail in U.S. Pat. Nos. 4,668,281, 4,667,668 and 4,805,811. Other suitable devices include dry powder inhalers such as the Rotahaler® (Glaxo), Discus® (Glaxo), SpirosTM inhaler (Dura

Pharmaceuticals), and the Spinhaler® (Fisons). Also suitable are devices which employ the use of a piston to provide air for either entraining powdered medicament, lifting medicament from a carrier screen by passing air through the screen, or mixing air with powder medicament in a mixing chamber with subsequent introduction of the powder to the patient through the mouthpiece of the device, such as described in U.S. Pat. No. 5,388,572.

Prior to use, dry powders are generally stored under ambient conditions, and preferably are stored at temperatures at or below about 25° C., and relative humidities (RH) ranging from about 30 to 60%. More preferred relative humidity conditions, e.g., less than about 30%, may be achieved by the incorporation of a desiccating agent.

The compositions of the invention are useful, when administered pulmonarily in a therapeutically effective amount to a mammalian subject, for treating or preventing any condition or disease associated with AAT-deficiency.

The terms “treat” and “treating” includes alleviating, ameliorating, halting, restraining, slowing or reversing the progression, or reducing the severity of pathological conditions described above.

AAT deficiency is a genetic condition that increases the risk of developing a variety of diseases including pulmonary emphysema (Laurell and Eriksson, Scand J Clin lab Inves, 1963. 15:132-140). It is caused by mutations in the AAT encoding gene (proteinase inhibitor (Pi) gene). Over 100 different allelic variants of the Pi genotype are recognized, of which 34 were found to be associated with a quantitative or functional deficiency of circulating AAT.

According to some embodiments, the dry powder composition is for use in treating Chronic Obstructive Pulmonary Diseases (COPD). A chronic obstructive pulmonary disease (COPD) is a disease state characterized by airflow limitation that is not fully reversible. The airflow limitation is usually progressive and associated with an abnormal inflammatory response of the lung to noxious particles or gases. Symptoms, functional abnormalities and complications of COPD can be attributed to this underlying phenomenon of abnormal inflammatory response and to processes related thereto.

The compositions of the invention may be further used for treating additional diseases or conditions that would benefit from AAT treatment e.g. diseases associated with overexpression of proteases that are inhibited by AAT. For example, overexpression or excessive activity of elastase may be treated with AAT.

AAT in aerosolized route may also be used as a treatment for cystic fibrosis (CF) patients who suffer from recurrent endobronchial infections and sinusitis. The major cause of morbidity and mortality among CF patients is lung diseases. CF patients carry a mutation in the CFTR gene, resulting in a malfunctioning CTFR protein, defective water and salt transport and the ensuing thick secretions in the lung. The membrane defect caused by the CFTR mutation leads to chronic lung inflammation and infection. In normal individuals, elastase secreted by neutrophils in response to infection is neutralized by AAT. AAT is known to penetrate into pulmonary tissue and exert its activity within this tissue. In patients with CF, however, the unregulated inflammatory response overwhelms the normal protease (elastase)/antiproteinase (AAT) balance. The abnormal cycle is destructively self-perpetuating and leads to the accumulation of elastase in the lung and ultimately to tissue damage, destruction of the lung architecture, severe pulmonary dysfunction and, ultimately, death. Supplemental AAT may reduce the deleterious effects associated with excessive amounts of elastase. It has been shown that inhalation of AAT by CF patients increased the AAT levels and decreased elastase activity levels, neutrophils, pro-inflammatory cytokines and numbers of Pseudomonas; in this study, however no effect on AAT lung function was observed (Matthias G. et al., ERJ Express. 2006. DOI: 10.1183/09031936.00047306).

The frequency of AAT treatment and the duration of each inhalation depends both on characteristics of the treated individual (age, weight, etc.) as well as on the characteristics of the pulmonary disease to be treated. According to certain embodiments, the duration of each inhalation is the duration of a single breath-taking, typically 1-2 seconds.

The dry powder may be used for treating exacerbation episodes of pulmonary diseases.

As used herein, the term “exacerbation” describes an increase in the severity of symptoms, which is mostly associated with a worsening of quality of life. Exacerbations are quite frequent in patients with chronic lung diseases in general and in AAT deficient patients in particular.

The pathology and pathophysiology of exacerbations are different from those resulting from the regular course of the disease. Pathology of exacerbation is based on sputum analysis and the pathophysiology is based on gas exchange measurements (see, e.g., GOLD global strategy for diagnosis, management, and prevention of chronic obstructive pulmonary disease, updated 2004). Symptom based definition of an exacerbation is made by measuring deterioration from baseline for at least 2 days of one or more of three major symptoms: dyspnea, sputum volume, and sputum color (see, e.g., Anthonisen NR, et al. Antibiotic therapy in exacerbations of chronic obstructive pulmonary disease. Ann Intern Med 1987; 106: 196-204).

Additional or alternative common way for defining exacerbations is “event based” method. In this method, occurrence of exacerbation event and its severity is categorized by change in concomitant medications/change in therapy. Treatment may include increased doses of long-acting β-2 adrenergic agonist inhalers, antibiotic agents, systemic corticosteroids, hospitalization, or combination thereof. This treatment change significantly differ the exacerbation event from the natural course/steady state of the respiratory disease.

According to certain embodiments, the treatment is administered at least once a week during an exacerbation. According to yet further embodiments, the treatment is administered at least twice a week, preferably at least once a day, more preferably at least twice a day during an exacerbation episode.

According to certain embodiments, administering AAT is performed in combination with at least one additional therapy. According to some embodiments, the additional therapy is selected from the group consisting of antibiotic therapy, administration of bronchodilators and anti-inflammatory therapy other than AAT therapy. According to other embodiments, the additional therapy is intravenous administration of AAT.

According to some embodiments, the dry powder compositions are delivered in doses of from 0.001 mg/day to 100 mg/day, in doses from 0.01 mg/day to 75 mg/day, or in doses from 0.10 mg/day to 50 mg/day. The composition may be administered every day or at intervals of one, two and three days as can be prescribed by a medical professional skilled in the Art.

The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein.

As used herein the term “about” in reference to a numerical value stated herein is to be understood as the stated value +/−10%, more preferably +/−5%, even more preferably +/−1%, and still more preferably +/−0.1% from the specified value.

### EXAMPLES

### Example 1

### Preparation of the Dry Powder Compositions

Various compounds were examined as potential excipients for dry powder compositions: Lactose, Mannitol, Trehalose, Glycine, Dipalmitoylphosphatidylcholine (DPPC), Ectoin, and their combinations

### Spray Drying Process

1. AAT was concentrated to 126 mg/ml by ultra-filtration (UF) system and sent to Pharmaterials, UK to assess spray drying of AAT formulations.

2. 24 ml of 126 mg/ml AAT solution was diluted in DI-water to 300 ml to achieve a final protein concentration of 10mg/ml. This was spray dried as a control, i.e. without any excipients.

3. For other experiments, excipient concentration in the solution was 10% w/w of AAT.

4. For experiments containing DPPC as excipient, DPPC was first dissolved in ethanol and added to the aqueous solution while continuously being stirred on magnetic stirrer.

5. Lab Plant spray-drier set with the following parameters:
Inlet temperature: 150° C.
Outlet temperature: 82° C.
Fan speed: 50 m³/hr.
Pump speed: 0.5 (Arbitrary Units)

### Results

The spray dried AAT formulations were analyzed, at Time=0 and after 1 month storage at 40° C./75% relative humidity (RH), by moisture content by STA (Differential Scanning calorimetry and Thermogravimetric Analysis), physical structure by X-ray powder diffraction (XRPD), interactions between AAT and the excipients by Raman Spectroscopy, particle size distribution by light scattering, AAT activity by Neutrophil Elastase inhibition, AAT antigen by Nephelometry and percentages of aggregates by SEC chromatography.

TABLE 1
Appearance of the different spray dried compositions at
T = 0 and T = 1 (40° C./75% RH)

| | Colour | Colour |
|---|---|---|
| Spray dried Formulation | (T = 0) | (T = 1 M at 40/75) |
| AAT | White fluffy powder | White fluffy powder |
| AAT + Lactose | White fluffy powder | Light yellow fluffy |
| | | powder* |
| AAT + Mannitol | White fluffy powder | White fluffy powder |
| AAT + Trehalose | White fluffy powder | White fluffy powder |
| AAT + Glycine | White fluffy powder | White fluffy powder |
| AAT + DPPC | White fluffy powder | White fluffy powder |
| AAT + Ectoin | White fluffy powder | White fluffy powder |
| AAT + Glycine + | White fluffy powder | White fluffy powder |
| Mannitol | | |
| AAT + DPPC + Mannitol | White fluffy powder | White fluffy powder |
| AAT + DPPC + Trehalose | White fluffy powder | White fluffy powder |

The table above shows that the spray dried process resulted in white and fluffy powders which in one case (in the presence of lactose as excipient) changed its color to light yellow, suggesting possible AAT degradation.

TABLE 2
Percentages of moisture content of the spray dried compositions
at T = 0 and T = 1 (40° C./75% RH)

| | | % Moisture | |
|---|---|---|---|
| | | content (w/w) | |
| | % Moisture | by STA at | |
| | (w/w) content | T = 1 | |
| | by STA at | (40° C./ | Variation/ |
| Spray dried Formulation | T = 0 | 75% RH) | change |
| AAT | 9.4 | 5.2 | Decrease |
| AAT + Lactose | 5.2 | 5.3 | Constant |
| AAT + Mannitol | 7.2 | 3.5 | Decrease |
| AAT + Trehalose | 6.4 | 4.2 | Decrease |
| AAT + Glycine | 8.0 | 2.9 | Decrease |
| AAT + DPPC | 2.8 | 4.5 | Increases |
| AAT + Ectoin | 4.6 | 5.01 | Slight |
| | | | Increase |
| AAT + Glycine + | 5.8 | 4.0 | Decrease |
| Mannitol | | | |
| AAT + DPPC + Mannitol | 3.7 | 2.8 | Decrease |
| AAT + DPPC + Trehalose | 6.5 | 4.9 | Decrease |

TABLE 3
diffraction results obtained by the
XRPD of the spray dried AAT powder

| | | Physical | |
|---|---|---|---|
| | | form by XRPD | |
| | Physical | at T = 1 M at | |
| | form by XRPD | 40° C./ | Variation/ |
| Spray dried Formulation | at T = 0 | 75% RH | Change |
| AAT | Amorphous | Amorphous | No change |
| AAT + Lactose | Amorphous | Amorphous | No change |
| AAT + Mannitol | Amorphous | Amorphous | No change |
| AAT + Trehalose | Amorphous | Amorphous | No change |
| AAT + Glycine | Amorphous | Amorphous | No change |
| AAT + DPPC | Amorphous | Amorphous | No change |
| AAT + Ectoin | Amorphous | Amorphous | No change |
| AAT + Glycine + | Amorphous | Amorphous | No change |
| Mannitol | | | |
| AAT + DPPC + Mannitol | Amorphous | Amorphous | No change |
| AAT + DPPC + Trehalose | Amorphous | Amorphous | No change |

It can be seen that AAT does not dry adequately without excipients and this could affect the aerosol performance of the powders.

To further characterize the dry powder a Raman Spectroscopy assay was performed for spray dried formulations at T=0 and T=1 (40° C./75% RH). AAT was not affected by spray drying process (data not shown). However, a spectrum from the formulation containing DPPC only as excipient suggests some interaction between AAT and DPPC.

Most of the formulations maintained the same spectra after 1 month storage at 40° C./75% RH. The AAT-Lactose spectra demonstrate significant changes from T=0 data and this indicates that AAT may be degraded in this formulation.

Next, the particles size of the dry powder was evaluated.

TABLE 4
Particle Size Distribution of the AAT Spray Dried formulations
at T = 0 and T = 1 (40° C./75% RH)

| | T = 0 | | | 1 Month | | |
|---|---|---|---|---|---|---|
| Spray Dried formulations | D₁₀(μm) | D₅₀(μm) | D₉₀(μm) | D₁₀(μm) | D₅₀(μm) | D₉₀(μm) |
| AAT | 3.125 | 4.363 | 10.477 | 3.163 | 6.695 | 13.390 |
| AAT + Lactose | 2.909 | 6.541 | 14.156 | 2.787 | 6.216 | 12.918 |
| AAT + Mannitol | 2.508 | 5.729 | 12.544 | 3.055 | 8.901 | 20.713 |
| AAT + Trehalose | 2.036 | 4.406 | 7.665 | 2.170 | 4.637 | 8.172 |
| AAT + Glycine | 2.446 | 5.094 | 9.534 | 2.765 | 5.465 | 9.822 |
| AAT + (DPPC) | 2.215 | 4.990 | 11.585 | 2.524 | 4.919 | 9.639 |
| AAT + Ectoin | 2.311 | 4.684 | 8.264 | 2.852 | 5.570 | 9.904 |
| AAT + Glycine + | 2.583 | 5.622 | 11.904 | 2.692 | 5.421 | 10.349 |
| Mannitol | | | | | | |
| AAT + DPPC + Mannitol | 2.966 | 8.467 | 20.47 | 3.055 | 8.901 | 20.413 |
| AAT + DPPC + Trehalose | 2.618 | 7.371 | 19.293 | 2.596 | 7.249 | 18.339 |

The results suggest that large particles with low density were formed when DPPC was used as excipient and small dense particles were formed when other excipients were used. Formulations containing DPPC occupied more volume and this suggests that particles with low density were produced. This phenomenon can lead to high aerosol performance of the powder as it affects the aerodynamic mass diameter of the particles so that even large, less dense particles, can reach the small airways of the lung.

Results after 1 month storage at 40° C./75% RH showed that the formulations were able to maintain the particle size distribution; particularly formulations containing trehalose, glycine, DPPC and ectoin.

TABLE 5
Summary results of AAT activity, total AAT by nephelometric
method and none monomeric forms percent (NMF %)
of Spray Dried formulations at T = 0.

| | | | | Ratio | |
|---|---|---|---|---|---|
| | | Active | | Active | |
| Experiment | | AAT | AAT by | AAT/AAT | |
| # | Excipient | mg/ml | Neph | by neph | NMF % |
| 1 | — | 6.456 | 7.000 | 0.922 | 8.79 |
| 2 | Lactose | 6.233 | 6.191 | 1.00 | 3.66 |
| 3 | Mannitol | 5.998 | 7.265 | 0.826 | 8.92 |
| 4 | Trehalose | 6.662 | 7.040 | 0.946 | 3.56 |
| 5 | Glycine | 6.133 | 6.912 | 0.887 | 4.38 |
| 6 | Ectoin | 6.533 | 6.869 | 0.951 | 3.34 |
| 7 | DPPC | 4.808 | 5.060 | 0.950 | 5.98 |
| 8 | Glycine/Mannitol | 6.284 | 6.454 | 0.974 | 6.91 |
| 9 | DPPC/Mannitol | 5.664 | 6.398 | 0.885 | 13.76 |
| 10 | DPPC/Trehalose | 4.632 | 6.042 | 0.767 | 20.58 |

The results obtained at T=0 show that most of the formulations maintained the specific activity of the protein (activity per antigen ratio) and in comparison to the AAT with no excipients, some formulations resulted in less aggregate percentages, e.g. Lactose, trehalose, glycine, ectoin and DPPC.

Overall, contrary to what is typically used in powder formulations for dry powder inhalers, lactose is not the best choice for AAT but the best possible formulations were found with trehalose and/or glycine and/or ectoin and/or DPPC as excipients.

### Example 2

### Testing the AAT Powder Formulations by Intratracheal Administration in Rats

Two formulations were prepared by spray dried process in order to assess the safety and systemic level of AAT in a powder formulation. The process was the same as detailed in Example 1. The formulations were administered once every other day for 7 days (4 treatments) by intratracheal administration in rats. As a control, a powder contains glycine and trehalose (1:1) was administered.

The powder formulations and their physical characterizations are shown in Table 6.

TABLE 6
Compositions and physical characterization
of the powder formulations

| | Control powder | | |
|---|---|---|---|
| | Glycine + | | |
| | Trehalose | | AAT + Glycine + |
| | (1:1) | AAT + DPPC | Trehalose |
| Compositions used | 50% Glycine + | 90.9% AAT + | 90.9 AAT + |
| for the spray dried | 50% Trehalose | 9.1% DPPC | 4.55% Glycine + |
| process | | | 4.55% Trehalose |
| D₅₀ (μm) based on | 4.1 | 11.5 | 4.8 |
| geometric particle | | | |
| size distribution | | | |
| (GPSD) analysis | | | |
| Residual moisture | 4.5 | 4.91 | 4.64 |
| (%) | | | |
| Assay | NR | 60 | 61 |
| (% w/w AAT in | | | |
| total powder) | | | |
| after powder | | | |
| reconstitution | | | |
| Fine partial | NR | 38.9 | 46.8 |
| fraction (FPF) | | | |
| by new generation | | | |
| impactor (NGI) | | | |
| (% of particles | | | |
| below 5 μm) | | | |

In Vivo Study

Seven weeks old Crl:CD(SD) Sprague-Dawley male rats (11 rats per group) were administered via the intratracheal route with two different powder formulations and one control formulation as described in Tables 7. The study included: safety analysis (main study), bronchia alveolar liquid analysis (BAL study) and toxicokinetic analysis (TK study), as detailed in Table 8.

TABLE 7
Test and Reference Item Identification

| | Reference Item | Powder | Powder |
|---|---|---|---|
| | (placebo powder) | Formulation 1 | Formulation 2 |
| | Group 1 | Group 2 | Group 3 |
| Identification | Control Powder | AAT + DPPC | AAT + Glycine + |
| | | | Trehalose |
| Physical | Yellow powder | White powder | White powder |
| Description | | | |
| Purity | 100% | 60% AAT | 60% AAT |
| Concentration | N/A | N/A | N/A |

TABLE 8
EXPERIMENTAL DESIGN

| | | | Number of Animals | | |
|---|---|---|---|---|---|
| | | | | | Toxi- |
| | | | Main | BAL | cokinetic |
| Group | | Dose | Study | Study | Study |
| No. | Test Material | Level | Males | Males | Males |
| 1 | Control | 1 | 5 | 3 | 3 |
| | | (mg/rat/day) | | | |
| 2 | Formulation 1 | 1* | 5 | 3 | 3 |
| | AAT + DPPC | (mg/rat/day) | | | |
| 3 | Formulation 2 | 1* | 5 | 3 | 3 |
| | AAT + Glycine + | (mg/rat/day) | | | |
| | Trehalose | | | | |

*All doses of AAT are equivalent to 0.6 mg/rat/day AAT

Animals were individually weighed at least once during the pretreatment period and on Days 4 and 8 during the treatment period. The main study groups (5 rats per each treatment) were subjected to complete necropsy examination, including evaluation of the carcass and musculoskeletal system; all external surfaces and orifices; cranial and external surfaces of the brain; and thoracic, abdominal and pelvic cavities with the associated organ and tissues. In addition the following organs were weighted, and were subjected to histopathology evaluation: body cavity, nasal, bronchus, carina, gross lesions/masses, larynx lung, lymph node, tracheobronchial, nasopharynx, pharynx and trachea.

The BAL study groups (3 rats for each treatment) were sacrificed and the lungs were collected for Broncho-alveolar Lavage (BAL). The BAL samples were analyzed for AAT and Urea concentrations (the urea was used as a normalization factor due to a different lavage volume). In parallel the AAT and the urea concentration were determined in the blood.

The toxicokinetic study groups (3 rats per each treatment) were tested for AAT concentrations in the blood at several time points after the first administration: 0, 2, 4, 8, 24 and 48 hours. In addition two additional bleeding points were added for the assessment of AAT accumulation in the blood.

Toxicokinetic parameters were estimated using Phoenix pharmacokinetic software.

### Results

All the rats have survived the four consecutive administrations of powder AAT over 7 days and no major pathological changes were observed. The lung weight, relatively to the body weight remained as in the control group (Table 9)

TABLE 9
The lung weight at the end of the study

| Group | Average BW (g) | Average Lung wt (g) | Lung/BW (%) |
|---|---|---|---|
| Control | 255.2 ± 19.8 | 1.1724 ± 0.0929 | 0.46 |
| Powder AAT | 275.2 ± 10.3 | 1.3210 ± 0.0328 | 0.48 |
| (DPPC) | | | |
| Powder AAT | 262.6 ± 17.6 | 1.2936 ± 0.0960 | 0.49 |
| (Gly + Tre) | | | |

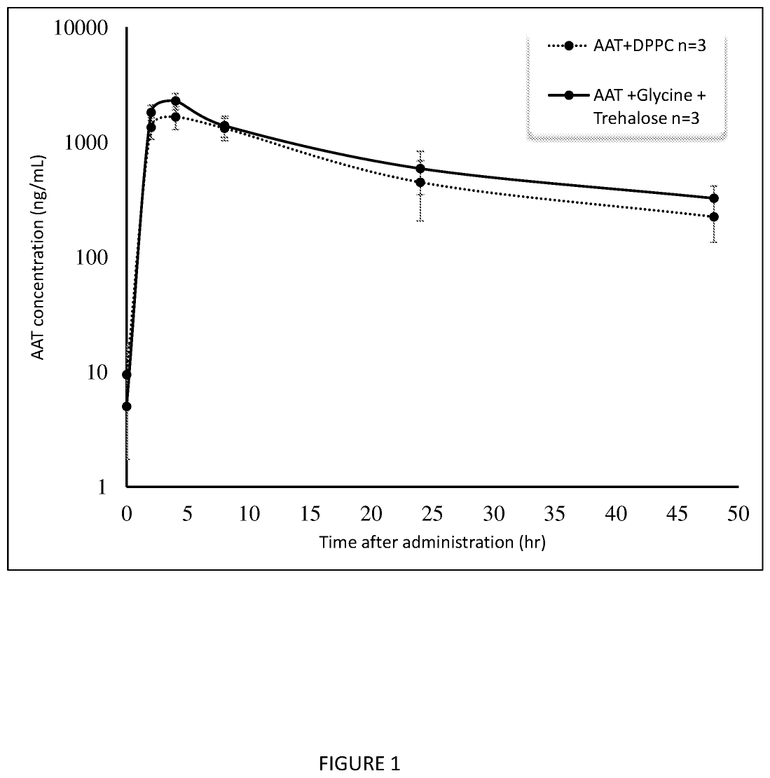

The Toxicokinetic (TK) results indicated that the AAT powder formulations have reached the blood stream as detected following 2 hrs post administration. The Tmax (the time need to reach the maximal AAT concentration in the blood) was 4 hrs. for both powder formulations, and the T1/2 was 16.5 and 17.6 hrs. for the DPPC and the Glycine+Trehalose formulations respectively. The calculated TK parameters are shown in Table 10 and the TK results are demonstrated in FIG. 1.

TABLE 10
TK parameters for the powder formulations

| | | Cmax | Tmax | AUC(0-t) | T½ | AUC(0-t)/D |
|---|---|---|---|---|---|---|
| Formulation | Day | (ng/ml) | (hr) | (hr*ng/ml) | (hr) | (hr*ng/ml/(mg)) |
| AAT + DPPC | 1 | 1660 ± 369 | 4 | 32500 ± 9950 | 16.5 | 54167 ± 16614 |
| AAT + Glycine | 1 | 2280 ± 995 | 4 | 40100 ± 19700 | 17.6 | 66778 ± 32863 |
| and Trehalose | | | | | | |

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A dry powder composition comprising at least 60% alpha-1 antitrypsin (AAT) dry powder and at least one excipient selected from the group consisting of Trehalose, Glycine, Dipalmitoylphosphatidylcholine (DPPC), and Ectoin, or a combination thereof, wherein at least 90% of the AAT is in a monomeric form.

2. The dry powder composition of claim 1, wherein the excipient is DPPC.

3. The dry powder composition of claim 1, wherein the excipient is a combination of Glycine and Trehalose.

4. The dry powder composition of claim 1, wherein the dry powder composition is suitable for delivery to the lung by inhalation.

5. The dry powder composition of claim 1, wherein the non-monomeric forms percent (NMF %) of AAT is less than 8%.

6. The dry powder composition of claim 5, wherein the (NMF %) of AAT is less than 6%.

7. The dry powder composition of claim 1, wherein the particle size distribution of the AAT dry powder, measured as D₅₀(μm), is less than 10 μm.

8. The dry powder composition of claim 1, wherein the particle size distribution of the AAT dry powder, measured as D₅₀(μm) after month of storage, is less than 10 μm.

9. The dry powder composition of claim 1, wherein the particle size distribution of the AAT dry powder, measured as D₉₀(μm) after month of storage, is less than 15 μm.

10. The dry powder composition of claim 1, wherein the dry powder composition is prepared by a spray-drying method.

11. The dry powder composition of claim 1, wherein the dry powder composition has a moisture content below 10% by weight.

12. The dry powder composition of claim 1, consisting of least 60% alpha-i antitrypsin (AAT) dry powder and at least one excipient selected from the group consisting of Trehalose, Glycine, Dipalmitoylphosphatidylcholine (DPPC), and Ectoin, and a combination thereof, wherein at least 90% of the AAT is in a monomeric form.

13. An inhalation device comprising the dry powder composition of claim 1.

14. The inhalation device of claim 13, wherein the dry powder has an emitted dose of at least 60%.

15. (canceled)

16. (canceled)

17. A method of treating a subject in need thereof, the method comprising pulmonarily administering to the subject a therapeutically effective amount of the dry powder of claim 1.

18. The method of claim 17, wherein the subject suffers from a condition or disease selected from the group consisting of: an AAT-deficiency, a disease associated with an AAT-deficiency, and a disease that would benefit from AAT administration.

19. The method of claim 18, wherein the disease is selected from the group consisting of: emphysema; chronic obstructive pulmonary disease (COPD); bronchiectasis; parenchymatic and fibrotic lung diseases or disorders; cystic fibrosis (CF), interstitial pulmonary fibrosis and sarcoidosis; tuberculosis; and lung diseases and disorders secondary to HIV.

20. The method of claim 18, wherein the subject suffers from an AAT deficiency, COPD, or CF.

21. (canceled)

22. (canceled)

23. The method of claim 17, wherein the subject is a human.

24. The method of claim of claim 17, further comprising administering to the subject an additional therapy selected from the group consisting of an antibiotic therapy and an anti-inflammatory therapy.

25. The method of claim 17, wherein the dry powder is administered at least once a week.
